(19) United States
(12) Patent Application Publication
(10) Pub. No.: US 2025/0230261 A1
(43) Pub. Date: Jul. 17, 2025

(54) COMPOSITIONS AND METHODS FOR ANTIGEN-SPECIFIC THERAPY

(71) Applicant: BODHI BIO LLC, Middletown, DE (US)

(72) Inventor: Animesh Sinha, Amherst, NY (US)

(73) Assignee: BODHI BIO LLC, Middletown, DE (US)

(21) Appl. No.: 18/848,994

(22) PCT Filed: Mar. 23, 2023
(86) PCT No.: PCT/US2023/016059

§ 371 Date: Sep. 20, 2024

Related U.S. Application Data
(60) Provisional application No. 63/322,702, filed on Mar. 23, 2022.
(60) Provisional application No. 63/322,699, filed on Mar. 23, 2022.

(51) Int. Cl. C07K 16/42; C07K 16/28

(52) U.S. Cl. CPC C07K 16/4241; C07K 16/2803; C07K 16/2809; C07K 16/2896

(57) ABSTRACT

A platform for therapy provides a multi-target therapeutic molecule comprising an anti-CD3 antigen binding fragment of an anti-CD3 antibody linked with a linker to one or more auto-antigen(s). The molecule can be further linked by another linker linking either an anti-CD3 antigen binding fragment of an anti-CD3 antibody or an auto-antigen to an anti-CD19 or anti-CD20 antibody or antigen-binding fragment thereof. The auto-antigens include Dsg1, Dsg3, bullous pemphigoid antigen 180, bullous pemphigoid antigen 230, MuSK, anti-phospholipase A2 Receptor, Factor VIII, one or more protein expressed by a viral vector administered to a subject for gene therapy, a DNA molecule carried by a gene therapy viral vector. A pharmaceutical composition comprises a disclosed multi-target therapeutic molecule is disclosed. Methods of treating diseases using the disclosed molecules are disclosed.

### TECHNICAL FIELD

This disclosure relates to a platform for using a molecule with a T-cell binding moiety and a B-cell binding moiety for therapy.

### BACKGROUND

The immune system is a powerful defense system for animals. Scientists are beginning to manipulate the immune system to make it attack antigens and cells in order to cure diseases. More is required to meet this goal.

### SUMMARY

In one aspect, this disclosure provides a multi-target therapeutic molecule as a platform for therapy. This multi-target therapeutic molecule is a chimeric molecule comprising a T-cell binding moiety (in some embodiments, the T-cell binding moiety binds to CD3) and a linker linking it to one or more auto-antigens (a B-cell binding moiety). In some embodiments, the targets of the multi-target therapeutic molecule are: 1) a T cell target molecule, and 2) one or more B cell target auto-antigen receptors. In some embodiments, the linker links an anti-CD3 antigen binding fragment of an anti-CD3 antibody (T cell target) to one or more auto-antigens (B cell targets). In some embodiments, the multi-target therapeutic molecule comprises another linker to link an anti-CD19 antibody, or antigen-binding fragment thereof (that also targets the B cell), to the T cell or the B cell targeting moieties. Other B cell targets may be linked.

In one aspect, the disclosure provides a multi-target therapeutic molecule as a platform for therapy for an autoimmune disease. The multi-target therapeutic molecule comprises an anti-CD3 antigen-binding fragment of an anti-CD3 antibody linked with a linker to one or more auto-antigen(s). In further embodiments, the auto-antigen is Dsg3 or Dsg1, or both. A pharmaceutical composition comprising a therapeutically effective amount of this multi-target therapeutic molecule is provided.

In another aspect, this disclosure provides a method of treating an autoimmune disease with known auto-antigen(s) in a patient by administering a pharmaceutical composition comprising a disclosed multi-target therapeutic molecule to said patient. In such case, the disclosed multi-target therapeutic molecule can be referred to herein also as “B-cell Selective Molecular Autoantigen-Receptor Targetors” (“B-SMAART”).

In some embodiments, a method of treating patients with Pemphigus vulgaris (PV), an autoimmune blistering skin disease where the B cell target auto-antigens, Desmoglein 3 (Dsg3) and Desmoglein 1 (Dsg1) are known, is provided. The method comprises administering a disclosed multi-target therapeutic molecule comprising an anti-CD3 antigen binding fragment of an anti-CD3 antibody linked with a linker to Dsg3 or Dsg1, or both.

In another aspect, the multi-target therapeutic molecule comprises an anti-CD3 antigen binding fragment of an anti-CD3 antibody linked with a linker to factor VIII. This multi-target therapeutic molecule can eliminate a patient's autoantibodies to Factor VIII through destruction of specific B cells that secrete anti-Factor VIII autoantibodies. This mechanism would restore the effectiveness of FVIII infusions. A pharmaceutical composition is provided that comprises a disclosed multi-target therapeutic molecule comprising an anti-CD3 antigen binding fragment of an anti-CD3 antibody linked with a linker to relevant factor VIII peptide stretches that contain relevant epitopes recognized by anti-Factor VIII autoantibodies. A method is disclosed to restore factor VIII therapy to a patient in need thereof comprising administering a disclosed pharmaceutical composition comprising a disclosed multi-target therapeutic molecule comprising an anti-CD3 antigen binding fragment of an anti-CD3 antibody linked with a linker to factor VIII.

In yet another aspect, the multi-target therapeutic molecule comprises an anti-CD3 antigen binding fragment of an anti-CD3 antibody linked with a linker to one or more protein expressed by a viral vector administered to a subject for gene therapy or linked to a DNA molecule carried by a viral vector administered to a subject for gene therapy. A pharmaceutical composition is provided that comprises a disclosed multi-target therapeutic molecule comprising an anti-CD3 antigen binding fragment of an anti-CD3 antibody linked with a linker to one or more protein expressed by a viral vector administered to a subject for gene therapy or linked to a DNA molecule of a viral vector administered to a subject for gene therapy. A method is disclosed to improve gene therapy in a patient in need thereof comprising administering a disclosed pharmaceutical composition comprising a disclosed pharmaceutical composition comprising a multi-target therapeutic molecule comprising an anti-CD3 antigen binding fragment of an anti-CD3 linked with a linker to one or more protein expressed by a viral vector administered to a subject for gene therapy or linked to a DNA molecule of a viral vector administered to a subject for gene therapy. The viral vector includes, without limitation, any adenovirus vector, adeno-associated vector, retroviral vector, herpes simplex viral vector, etc. The protein expressed by the viral vector includes viral capsid protein.

In yet another aspect, this disclosure provides a method of identifying an auto-antigen or auto-antigens of an autoimmune disease comprising screening a multiplexed platform comprising auto-antigen/epitope protein microarray with an autoantibody associated with the autoimmune disease.

The “multi-target” therapeutic molecule can have two targets or more than two targets.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1A (patient PV327), FIG. 1B (patient PV102), and FIG. 1C (patient PV114) show IgG reactivity in a longitudinal analysis for 3 patients. The y axis shows fold change in expression levels of the various autoantibodies in the blood. The X-axis shows data point for same patient in different phases of disease: A=active disease; LTR=long term remission (>6 m).

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. “Effective amount,” “prophylactically effective amount,” or “therapeutically effective amount” refers to an amount of an agent or composition that provides the desired biological, therapeutic, and/or prophylactic result. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations.

As used herein, a “patient” and a “subject” are interchangeable terms and may refer to a human patient/subject, a dog, a cat, a non-human primate, etc.

The term “antibody fragment,” “antigen-binding fragment of an antibody,” and the like are known in the art.

The term “antibody fragment,” “antigen-binding fragment of an antibody,” and the like can, for example, refer to a fragment of an antibody that retains the ability to bind to a target antigen. Such fragments include, e.g., a single chain antibody, a single chain Fv fragment (scFv), a Fd fragment, a Fab fragment, a Fab′ fragment, or a F(ab′)2 fragment. An scFv fragment is a single polypeptide chain that includes both the heavy and light chain variable regions of the antibody from which the scFv is derived. In addition, intrabodies, minibodies, triabodies, and diabodies are also included in the definition of an antigen-binding fragment of an antibody. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283. An antigen-binding fragment can also include the variable region of a heavy chain polypeptide and the variable region of a light chain polypeptide. An antigen-binding fragment can comprise the CDRs of the light chain and heavy chain polypeptide of an antibody.

The term “antibody fragment,” “antigen-binding fragment of an antibody,” and the like also can include, e.g., single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079. The term “antibody fragment” also includes single domain antibodies comprising two V_{H} domains with modifications such that single domain antibodies are formed.

The term ‘an antigen-binding fragment” of an antibody can also include the entire Fc tail of an antibody.

The terms “self-antigen” and “auto-antigen” are used interchangeably herein.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should be considered to include the end points 5 and 10.

The term “CD3” refers to cluster of differentiation 3 and is a protein complex and T cell co-receptor.

The term “CD19” refers to cluster of differentiation 19, a protein that is also known as B-Lymphocyte Surface Antigen B4, T-Cell Surface Antigen Leu-12 and CVID3.

The term “factor VIII” (FVIII) refers to an essential blood-clotting protein. Defects in FVIII results in Hemophilia A in a human patient.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Autoimmune Diseases

Autoimmune disease, prevalent in the population, is a major healthcare burden. An autoimmune disease is when a host's immune system attacks one or more self-antigens (auto-antigens). In the majority of autoimmune diseases the target auto-antigen(s) are not known. There are over 100 known human autoimmune diseases, affecting between 5-10% of the population. Autoimmune diseases are the 2^{nd} or 3^{rd} leading cause of morbidity and mortality and cost the US healthcare system over $100 billion annually. Lupus, pemphigoid, myasthenia gravis, multiple sclerosis, type 1 diabetes, and pemphigus vulgaris are just some examples of autoimmune diseases. Treatment options for autoimmune diseases are limited and largely non-specific or symptom oriented. No true cures are available and there is no consensus treatment guidelines. Targeted, individualized therapies are lacking.

Pemphigus is a group of IgG-mediated autoimmune diseases of stratified squamous epithelia, such as the skin and oral mucosa, in which acantholysis (the loss of cell adhesion) causes blisters and erosions. Pemphigus has three major subtypes: pemphigus vulgaris, pemphigus foliaceus and paraneoplastic pemphigus.

Pemphigus vulgaris (PV) is a potentially life-threatening autoimmune blistering skin disease, characterized by intraepithelial (suprabasalar) acantholysis, which is a loss of cell-cell adhesion. Quite a bit is known about PV. HLA genetic predisposition (HLA DRB1*0402 and DQB1*0503) is known; T cell (Th₂ driven) and B cell subsets (producing IgG4 autoantibodies) are known. And the primary autoantibody targets (autoantigens)—Desmoglein (Dsg)-3 and Desmoglein-1—are known.

Dsg3 and Dsg1 are keratinocyte-associated cell surface proteins relevant to cell-cell adhesion. Anti-Dsg3 and anti-Dsg1 autoantibodies can be detected in human PV patients and can be followed by ELISA. The titers roughly correlate with disease activity and serves as disease biomarkers.

Current and proposed treatments of PV include general immunosuppression with, for example, steroids; immunoglobulin-focused therapy, such as intravenous IG or FcRn blockade; B-cell targeted therapies, such as anti-CD20 molecules, BTK inhibitors, or BAFF inhibitors; and antigen-specific therapies.

Currently proposed antigen-specific therapy makes use of Chimeric Auto-Antibody Receptor T (CAAR-T) cells, an adaptation of the CAR-T cell strategy. CAAR-T cells are T-cells engineered to express autoantigen-based chimeric immunoreceptors. This platform directs T cells to kill autoreactive B lymphocytes through the specificity of the B cell receptor (BCR), without the requirement for T-cells (autologous or allogeneic). For PV, engineered human T cells expressing the PV autoantigen Dsg3 exhibit specific cytotoxicity against cells expressing anti-Dsg3 BCRs in vitro and specifically eliminate Dsg3-specific B cells in vivo in a PV mouse model.

Major hurdles, however, are associated with such cell-based therapies. These hurdles include:
complicated manufacturing process, involving harvesting of autologous T cells, engineering of cells, reinfusing cells and failing productions;
treatment time lag from start to finish;
complex patient referral pathway;
accredited CAAR T cell specialty centers and trained staff are needed;
potential for significant, life-threatening adverse effects;
potential for long lived, permanence of therapy;
inability to tune down therapy;
inability to readily adapt to multiple target therapy;
inability to readily personalize to individual patients;
inability to readily adapt to evolving autoimmune response in a given patient;
exorbitant costs;
commercial scalability challenges; and
complicated payer policies.

### Hemophilia

Hemophilia A patients can be treated successfully with factor VIII. However, 5-30% of patients with hemophilia A (of all severities) develop inhibitory anti-factor VIII antibodies (inhibitors) following replacement therapy.

### Gene Therapy Vectors

Gene therapy holds enormous promise. However, a patient's immune system can be a hindrance to gene therapy. Viral capsids, viral-vector DNA (also referred herein as a DNA molecule carried by a viral vector), and even the transgene products themselves may be recognized as foreign by the immune system. Immunity against viral capsids, viral-vector DNA (also referred herein as a DNA molecule carried by a viral vector), and transgene products can limit the efficacy and restrict dosing of gene therapy.

### Molecules, Compositions, and Methods

This disclosure provides a multi-target therapeutic molecule as a platform for therapy. This molecule is a chimeric molecule comprising a T-cell binding moiety (in some embodiments, the T-cell binding moiety binds to CD3) and a linker linking the T-cell binding moiety to one or more auto-antigens (B-cell binding moiety). In some embodiments, the T-cell binding moiety binds to CD3.

The goal of the therapeutic platform is to simultaneously bind to a naïve T-cell (in some embodiments, to CD3⁺ T cell) and to one or more B-cells bearing the proper B cell receptor(s) for the auto-antigen(s), both attached on the same molecule. By bridging the two cells via this multi-target therapeutic molecule, the T-cell is able to mediate destruction of the auto-antigen-targeted B cell.

The “multi-target” therapeutic molecule can have two or more targets. In some embodiments, the “multi-target” therapeutic molecule is a “dual-target” therapeutic molecule. As used herein, the term “multi-target” therapeutic molecule includes “dual-target” therapeutic molecule. A dual-target therapeutic molecule has one T-cell binding moiety (in some embodiments, the T-cell binding moiety binds to CD3) and a linker linking the T-cell binding moiety to one auto-antigen (a B-cell binding moiety). A multi-target therapeutic molecule can have more than one auto-antigens and/or another protein linked to it, such as an anti-CD19 moiety or an anti-CD20 moiety.

In one aspect, the disclosure provides a multi-target therapeutic molecule as a platform for therapy for an autoimmune disease. The multi-target therapeutic molecule comprises an anti-CD3 antigen binding fragment of an anti-CD3 antibody linked with a linker to one or more auto-antigen(s) that are known to be auto-antigen(s) of an autoimmune disease in a subject. In some embodiments, the auto-antigen is Desmoglein 3 (Dsg3) or Desmoglein 1 (Dsg1). Dsg3 and Dsg1 are known B cell target auto-antigens of patients with Pemphigus vulgaris (PV), an autoimmune blistering skin disease. In further embodiments, the multi-target therapeutic molecule comprises another linker to link an anti-CD19 antibody, or antigen-binding fragment thereof (that also targets the B cell) to the T-cell (in some embodiments, anti-CD3) or the B-cell (in some embodiments, auto-antigen) targeting moieties.

In some embodiments, instead of Dsg3 and Dsg1, other known auto-antigens of autoimmune diseases can be a part of the multi-target therapeutic molecule. In some embodiments, a disclosed multi-target therapeutic molecule comprises bullous pemphigoid antigen 180 and/or bullous pemphigoid antigen 230. Bullous pemphigoid antigen 180, Bullous pemphigoid antigen 230 are known auto-antigens of patients with bullous pemphigoid. In some embodiments, a disclosed multi-target therapeutic molecule comprises muscle specific tyrosine kinase (MuSK). MuSK is a known auto-antigen of patients with myasthenia gravis (MG). In other embodiments, a disclosed multi-target therapeutic molecule comprises an anti-phospholipase A2 Receptor (PLA2R). PLA2R is a known auto-antigen of patients with membranous nephropathy (MNEP), a disorder where the body's immune system attacks the filtering membranes in the kidney. These membranes clean waste products from the blood.

In another aspect, this disclosure provides a pharmaceutical composition comprising an effective amount of a disclosed multi-target therapeutic molecule.

In another aspect, this disclosure provides a method of treating an autoimmune disease with known auto-antigen(s) in a patient by administering a pharmaceutical composition comprising a disclosed multi-target therapeutic molecule to said patient. In such case, the disclosed multi-target therapeutic molecule can be referred to herein also as “B-cell Selective Molecular Autoantigen-Receptor Targetors” (“B-SMAART”).

In some embodiments, a method of treating a patient with Pemphigus vulgaris (PV), an autoimmune blistering skin disease where the B cell target auto-antigens, Desmoglein 3 (Dsg3) and Desmoglein 1 (Dsg1) are known, is provided. The method comprises administering a disclosed pharmaceutical composition comprising a disclosed multi-target therapeutic molecule comprising an anti-CD3 antigen binding fragment of an anti-CD3 antibody linked with a linker to Dsg3 or Dsg1. In further embodiments, the disclosed multi-target therapeutic molecule comprising an anti-CD3 antigen binding fragment of an anti-CD3 antibody linked with a linker to Dsg3 or Dsg1 and is further linked to an anti-CD19 or anti-CD20 antibody or antigen-binding fragment thereof to either the anti-CD3 antigen binding fragment of an anti-CD3 antibody or to Dsg3 or Dsg1.

In some embodiments, a method is provided of treating a patient with bullous pemphigoid, an autoimmune blistering skin disease that causes large, fluid-filled blisters. The method comprises administering a disclosed pharmaceutical composition comprising a disclosed multi-target therapeutic molecule comprising an anti-CD3 antigen binding fragment of an anti-CD3 antibody linked with a linker to bullous pemphigoid antigen 180 or bullous pemphigoid antigen 230. In further embodiments, the disclosed multi-target therapeutic molecule comprising an anti-CD3 antigen binding fragment of an anti-CD3 antibody linked with a linker to bullous pemphigoid antigen 180 or bullous pemphigoid antigen 230 and is further linked to an anti-CD19 or anti-CD20 antibody or antigen-binding fragment thereof to either the anti-CD3 antigen binding fragment of an anti-CD3 antibody or to bullous pemphigoid antigen 180 or bullous pemphigoid antigen 230.

In some embodiments, a method is provided of treating a patient with a sub-type of myasthenia gravis, an autoimmune, neuromuscular disease that causes weakness in the skeletal muscles. The method comprises administering a disclosed pharmaceutical composition comprising a disclosed multi-target therapeutic molecule comprising an anti-CD3 antigen binding fragment of an anti-CD3 antibody linked with a linker to muscle specific tyrosine kinase. In further embodiments, the disclosed multi-target therapeutic molecule comprising an anti-CD3 antigen binding fragment of an anti-CD3 antibody linked with a linker to muscle specific tyrosine kinase and is further linked to an anti-CD19 or anti-CD20 antibody or antigen-binding fragment thereof to either the anti-CD3 antigen binding fragment of an anti-CD3 antibody or to muscle specific tyrosine kinase.

In some embodiments, a method is provided of treating a patient with membranous nephropathy (MNEP), a disorder where the body's immune system attacks the filtering membranes in the kidney. These membranes clean waste products from the blood. The method comprises administering a disclosed pharmaceutical composition comprising a disclosed multi-target therapeutic molecule comprising an anti-CD3 antigen binding fragment of an anti-CD3 antibody linked with a linker to an anti-phospholipase A2 Receptor (PLA2R). In further embodiments, the disclosed multi-target therapeutic molecule comprising an anti-CD3 antigen binding fragment of an anti-CD3 antibody linked with a linker to PLA2R and is further linked to an anti-CD19 or anti-CD20 antibody or antigen-binding fragment thereof to either the anti-CD3 antigen binding fragment of an anti-CD3 antibody or to PLA2R.

In other aspects, the multi-target therapeutic molecule comprises an anti-CD3 antigen binding fragment of an anti-CD3 antibody linked with a linker to factor VIII. This multi-target therapeutic molecule can destroy the antibodies and B cells that neutralize the FVIII clotting factor and thus restore the effectiveness of FVIII infusions to a patient in need thereof (such as a patient with hemophilia A). In further embodiments, the disclosed multi-target therapeutic molecule comprising an anti-CD3 antigen binding fragment of an anti-CD3 antibody linked with a linker to factor VIII and is further linked to an anti-CD19 antibody or antigen-binding fragment thereof to either the anti-CD3 antigen binding fragment of an anti-CD3 antibody or to factor VIII.

A pharmaceutical composition is provided that comprises a disclosed multi-target therapeutic molecule comprising an anti-CD3 antigen binding fragment of an anti-CD3 antibody linked with a linker to factor VIII, with or without further linkage to an antibody or antigen-binding fragment thereof to CD19 or CD20.

A method is disclosed to restore factor VIII effectiveness to a patient in need thereof comprising administering a disclosed pharmaceutical composition comprising a disclosed multi-target therapeutic molecule comprising an anti-CD3 antigen binding fragment of an anti-CD3 antibody linked with a linker to factor VIII, with or without further linkage to an antibody or antigen-binding fragment thereof to CD19 or CD20.

In yet another aspect, the multi-target therapeutic molecule comprises an anti-CD3 antigen binding fragment of an anti-CD3 antibody linked with a linker to one or more protein expressed by a viral vector administered to a subject for gene therapy or linked to a DNA molecule of a viral vector administered to a subject for gene therapy. In further embodiments, the disclosed multi-target therapeutic molecule comprising an anti-CD3 antigen binding fragment of an anti-CD3 antibody linked with a linker to one or more protein expressed by a viral vector administered to a subject for gene therapy or linked to a DNA molecule of a viral vector administered to a subject for gene therapy and is further linked to an anti-CD19 or anti-CD20 antibody or antigen-binding fragment thereof to either the anti-CD3 antigen binding fragment of an anti-CD3 antibody or to one or more protein expressed by a viral vector administered to a subject for gene therapy or linked to a DNA molecule of a viral vector administered to a subject for gene therapy.

A pharmaceutical composition is provided that comprises a disclosed multi-target therapeutic molecule comprising an anti-CD3 antigen binding fragment of an anti-CD3 antibody linked with a linker to one or more proteins expressed by a viral vector administered to a subject for gene therapy or linked to a DNA molecule of a viral vector administered to a subject for gene therapy, with or without further linkage to an antibody or antigen-binding fragment thereof to CD19 or CD20.

A method is disclosed to improve gene therapy in a patient in need thereof comprising administering a disclosed pharmaceutical composition comprising a disclosed pharmaceutical composition comprising a multi-target therapeutic molecule comprising an anti-CD3 antigen binding fragment of an anti-CD3 antibody linked with a linker to one or more protein expressed by a viral vector administered to a subject for gene therapy or linked to a DNA molecule carried by a viral vector administered to a subject for gene therapy, with or without further linkage to an antibody or antigen-binding fragment thereof to CD19 to CD20. Given that gene therapy often is given only once, in certain embodiments, this method is used at about the same time, or shortly before or shortly after, as the gene therapy itself. In some embodiments, the disclosed method improves gene therapy efficacy and allows normal dosing of gene therapy.

The viral vector for gene therapy includes, without limitation, adenovirus vector, adeno-associated vector, retroviral vector, herpes simplex viral vector, etc. The protein expressed by the viral vector includes a viral capsid protein. The protein expressed by the viral vector includes fragments thereof, mutants thereof or variants thereof, but those that still bind to the auto-antibodies. The viral DNA (a DNA molecule carried by a viral vector) can be the entire viral genome or mutants/variants thereof, a part of the viral genome or mutants/variants thereof, but those that still bind to the auto-antibodies.

In further embodiments, epitope mapping of the various proteins of the vector, such as an adeno-associated vector (AAV) reveals frequently targeted epitope or region on the viral protein that elicits an auto-immune response. That epitope or region can then be used as part of the disclosed multi-target therapeutic molecule.

In some embodiments, a linker links an anti-CD3 antigen binding fragment of an anti-CD3 antibody to one or more auto-antigens and another linker links the anti-CD3 antigen binding fragment of an anti-CD3 antibody or the one or more auto-antigens to an anti-CD19 or anti-CD20 antibody or antigen-binding fragment thereof.

The disclosure includes using a full-length antibody to CD3 as part of the molecule (or mutant/variant thereof) as well. However, a smaller antigen-binding fragment than a whole antibody can be easier to accommodate in size and leads to less steric hindrance.

In some embodiments, the anti-CD3 antigen binding fragment of an anti-CD3 antibody is a single chain antibody. In some embodiments, the anti-CD3 antigen binding fragment of an anti-CD3 antibody is a Fab fragment, a Fab′ fragment, or a F(ab′)2 fragment. In certain embodiments, the anti-CD3 antigen binding fragment of an anti-CD3 antibody is a a single chain Fv fragment. In some embodiments, the anti-CD3 antigen binding fragment of an anti-CD3 antibody is a fragment of an IgG molecule.

In some embodiments, the anti-CD19 or anti-CD20 antigen binding fragment of an anti-CD19 or anti-CD20 antibody is a single chain antibody. In some embodiments, the anti-CD19 or anti-CD20 antigen binding fragment of an anti-CD19 or anti-CD20 antibody is a Fab fragment, a Fab′ fragment, or a F(ab′)2 fragment. In certain embodiments, the anti-CD19 or anti-CD20 antigen binding fragment of an anti-CD3 antibody is a a single chain Fv fragment. In some embodiments, the anti-CD19 or anti-CD20 antigen binding fragment of an anti-CD3 antibody is a fragment of an IgG molecule.

In some embodiments, a disclosed multi-specific molecule comprises an antibody to CD3, CD19, or CD20 with reduced or no fucose moieties. The alteration involves having fucose moieties removed from the antibody moieties of the disclosed multi-specific molecule or have the antibody made such that low or no fucose is added to it. Low or no fucose on the antibody moieties of the disclosed multi-specific molecule can be accomplished by methods known in the art, including, without limitation, producing the disclosed multi-specific molecule in specific cell lines that lack or are deficient in enzyme(s) responsible for fucosylation, or in the pathway for generating fucose, or enzymatically removing fucose moieties by treating the disclosed multi-specific molecule with glycosidase and glycosynthase enzymes.

In some embodiments, one or more linkers link an anti-CD3 antigen binding fragment of an anti-CD3 antibody to multiple auto-antigens.

The autoantigen can be a full-sized protein or smaller polypeptides (or variants/mutants of either a full-sized protein or smaller polypeptide) but still bind to its antibody or antigen-binding fragment thereof. In some embodiments, the size of the auto-antigen is optimized for binding to its cognate B-cell receptor but does not get bound by a patient's circulating auto-antibodies to the auto-antigen. The known auto-antigens are known in the art and can be obtained by, for example, recombinant DNA technology or other methods known in the art. The auto-antigens may also be purchased or gifted.

The antibodies to CD3, CD19, CD20 are also known in the art or can be obtained or generated by routine laboratory techniques. CD3, CD19 and CD20 proteins are known and can be obtained or generated by routine laboratory techniques.

Any suitable linker can be used. The linker is one that generally makes a covalent bond or covalent bonds with the protein(s) or DNA. The linker is one that links two protein molecules or fragments together or a protein molecule and a DNA molecule or fragments thereof.

In some embodiments, the linker is a peptide. A peptide linker can be composed of small, non-polar (e.g., Gly) or polar (e.g., Ser or Thr) amino acids. The linker can be poly-glycine or poly-glycine with one or more Ser and/or Thr. A peptide linker can be generated as part of the multi-target therapeutic molecule by recombinant DNA technology.

In other embodiments, the linker is a chemical non-peptide moiety. Proteins are typically cross-linked in a chemical reaction involving a cross-linker and side chains of amino acids. The reactivity of amino groups, thiols and carboxylic acids, render them as prime targets for cross-linking. The cross-linker can be a molecule with two reactive groups on either end, separated by a spacer. These reactive groups can target either primary amino groups (found in the side chain of lysine and at the protein N-terminus) or thiols (cysteine side chain). A small molecule, 1-Ethyl-3-(3-dimethylaminopropyl)carbodiimide (EDC), can be used to activate carboxylic acids (aspartate, glutamate, protein C-terminus) to cross-link with amines (lysine, protein N-terminus). This directly cross-links atoms of the protein(s) with each other in a “zero-length” cross-link. Other cross-linkers have been synthesized by introducing N-hydroxyphthalimide, hydroxybenzotriazole, and 1-hydroxy-7-azabenzotriazole as leaving groups instead of the commonly used N-hydroxysuccimidyl moiety. Other examples of chemical linkers include, without limitation, Bis(sulfosuccinimidyl) suberate (BS3), polyethylene glycol (PEG), which can be used as a single or branched chained moiety in a pegylation reaction, block sulfhydryls, such as N-Ethylmaleimide (NEM) and S-methyl methanesulfonothioate (MMTS), N-Succinimidyl-S-acetylthioacetate (SATA), and 2-Iminothiolane-HCl (Traut's reagent). In some embodiments, the cross linker is an acid-sensitive cis-aconityl group, such as, for example, cis-aconitic anhydride. Diener, E., Diner, U., Sinha, A., Xie, S., and Vergidis, R. 1986, Science 231(4734): 148-150; Diener, U., Diener, E., Sinha, A., Xie, S., and Vergidis, R. 1986. Selective suppression of murine lymphocyte function by daunomycin conjugated via an acid sensitive spacer to target specific carriers. In: Mediators of Immune Regulation and Immunotherapy (S. K. Singhal and T. L. Delovitch, Eds.), Elsevier Science Publications, Amsterdam, p. 177-181. These chemical non-peptide linkers are attached to proteins chemically by reactions known in the art. See, e.g., Id.

The auto-antigen can be an antigenic determinant of the auto-antigen, one that maintains the ability to bind to its B cell receptor. The auto-antigen, or its antigenic determinant, can be part of a fusion protein. The auto-antigen can be full-length or can be fragments and can be variants or mutants of either the full-length auto-antigen or fragments of the auto-antigen, so long as the auto-antigen is still recognized by the auto-antibody direct to it.

In some embodiments, the anti-CD3 antibody, or an antigen (CD3) binding fragment thereof, is any protein molecule that binds CD3, which is found on all T-cells. The anti-CD3 antibody, or an antigen (CD3) binding fragment thereof, binds to T-cells expressing CD3 on their surface. Generating fragments of a protein by, for example, recombinant DNA technology, is routine in the art.

In some embodiments, the auto-antigen is Dsg3. In other embodiments, the auto-antigen is Dsg1. The auto-antigen can be an antigenic determinant of Dsg3 or Dsg1 that maintains the ability to bind to its B-cell receptor. In further embodiments, the multi-target therapeutic molecules comprises anti-CD3 antibody, or an antigen-binding fragment thereof, with a linker to Dsg3 and to Dsg1. In further embodiments, the multi-target therapeutic molecules comprises an anti-CD3 antigen binding fragment of an anti-CD3 antibody (in certain embodiments, the antigen binding fragment is an F_{AB} domain to CD3), with a linker to Dsg3 and/or to Dsg1 and to anti-CD20 antibody, or an antigen-binding fragment thereof.

The auto-antigen(s) can be chimeric/fusion proteins. The auto-antigen(s) on the disclosed multi-target therapeutic molecule binds to B-cells expressing on their surface B-cell receptors to the auto-antigens, such as to Dsg3 and to Dsg1. The auto-antigen on the disclosed multi-target therapeutic molecule serves as lure to attract auto-reactive B cells expressing auto-antigen specific B cell receptors on their surface. The anti-CD3 antibody attracts killer T cells and brings them in close proximity to the autoreactive B cells to facilitate their elimination. The anti-CD19 moiety could further serve to bring T cells and B cells in close proximity. Generating fragments of a protein or fusion protein by, for example, recombinant DNA technology, is routine in the art.

In another aspect, this disclosure provides a pharmaceutical composition comprising a disclosed multi-target therapeutic molecule.

This disclosure provides a method of treating an autoimmune disease with known auto-antigen(s) in a patient comprising administering a pharmaceutical composition comprising a disclosed multi-target therapeutic molecule to said patient.

A method of treating PV in a patient in need thereof is provided, comprising administering a disclosed multi-target therapeutic molecule to said patient.

Autoimmune diseases with known auto-antigens include, without limitation, pemphigus, pemphigoid, myasthenia gravis (such as, for example and without limitation, acetylcholine receptors (AChRs), muscle-specific kinase (MuSK) and low-density lipoprotein receptor-related protein 4 (LRP4)), Type 1 Diabetes (such as, for example and without limitation, insulin, glutamic acid decarboxylase, and protein tyrosine phosphatase), and multiple sclerosis (myelin).

Similar to bi-specific antibodies this approach facilitates a “synapse” between T cells and antigen-specific B cells to facilitate T cell mediated, antigen-specific elimination of autoreactive B-lymphocytes.

The disclosure provides a highly specific, potentially less toxic strategy to create a “targeted bullet” for the treatment of autoimmune diseases. The disclosed multi-target therapeutic molecule can be:
antigen-specific (multiple auto-antigens could be linked);
can be modified for any autoimmune disease where the antigen target are known; and
can be individualized for a given patient.

The disclosed molecule, method and system do not require harvesting of autologous patient lymphocytes; do not require genetic engineering of patient T cells; and do not require reinfusion of autologous T cells.

Methods of making the disclosed multi-target therapeutic molecules are known in the art. For example, recombinant DNA technology can be used to make the separate protein molecules; chemical reactions to link proteins to each other with a linker or a protein and a DNA together with a linker are known and are used to link these moieties to each other. In case of a peptide linker, the multi-target therapeutic molecule, or at least part of it, can be made as a large fusion protein.

In yet another aspect, this disclosure provides a method of identifying auto-antigenic targets/epitopes of an autoimmune disease comprising screening a multiplexed platform comprising auto-antigen/epitope protein microarray with an autoantibody associated with the autoimmune disease. The auto-antigen/epitope protein microarray can be prepared by methods known in the art and the microarray can have any protein molecules or fragments thereof, including those protein molecules suspected to be the auto-antigens or epitopes thereof.

### Formulating and Administering Compositions

The disclosed composition may be administered to a subject in need thereof by any suitable mode of administration, any suitable frequency, and at any suitable, effective dosage.

The composition for use in a disclosed method may be in any suitable form and may be formulated for any suitable means of delivery.

In some embodiments, the disclosed composition is provided in a form suitable for injection, such as subcutaneous, intramuscular, intravenous, intraperitoneal, or any other appropriate route of injection. In some embodiments, compositions for injection are provided in sterile and/or non-pyrogenic form and may contain preservatives and/or other suitable excipients, such as sucrose, sodium phosphate dibasic heptahydrate or other suitable buffer, a pH-adjusting agent such as hydrochloric acid or sodium hydroxide, and polysorbate 80 or other suitable detergent.

When provided in solution form, in some embodiments, the composition for use in a disclosed method is provided in a glass or plastic bottle, vial or ampoule, any of which may be suitable for either single or multiple use. The bottle, vial or ampoule containing the disclosed composition may be provided in kit form together with one or more needles of suitable gauge and/or one or more syringes, all of which preferably are sterile. Thus, in certain embodiments, a kit is provided comprising a liquid solution as described above, which is packaged in a suitable glass or plastic bottle, vial or ampoule and may further comprising one or more needles and/or one or more syringes. The kit may further comprise instruction for use.

The disclosed composition can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the disclosed composition comprises at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N.J. 1991) or other standard pharmaceutical science texts, such as the Handbook of Pharmaceutical Excipients (Shesky et al. eds., 8th ed. 2017).

In some embodiments, the disclosed composition can be administered intravenously, intraperitoneally or intramuscularly, but other suitable routes of administration are also possible.

Water may be used as a carrier and diluent in the composition. The use of other pharmaceutically acceptable solvents and diluents in addition to or instead of water is also acceptable.

Large macromolecules that are slowly metabolized, such as proteins, polysaccharides, polylactic acids, polyglycolic acids, polymeric amino acids, copolymers of amino acids, can also be used as carrier compounds for the composition. Pharmaceutically acceptable carriers in therapeutic compositions may additionally contain liquids, such as water, saline, glycerol or ethanol. Moreover, the said compositions may further comprise excipients, such as wetting agents or emulsifiers, buffering substances, and the like. Such excipients include, among others, diluents and carriers conventional in the art, and/or substances that promote penetration of the active compound into the cell, for example, DMSO, as well as preservatives and stabilizers.

The composition for use in a disclosed method may be presented in various dosage forms depending on the object of application; in particular, it may be formulated as a solution for injections.

The composition for use in a disclosed method may be administered systemically. Suitable routes of administration include, for example, by parenteral administration, such as intravenous, intraperitoneal administration. However, depending on a dosage form, the disclosed composition may be administered by other routes.

The disclosed composition can be co-administered with another appropriate agent or therapy.

### EXAMPLES

For this invention to be better understood, the following examples are set forth. These examples are for purposes of illustration only and are not to be construed as limiting the scope of the invention in any manner.

### Example 1 Assessment of Auto-Ab Specificity in PV Patients—Development of a Multiplexed Platform to Comprehensively Identify Autoantigens in an Autoimmune Disease

Multiplexed protein microarrays were used to probe PV patient or negative control sera.

Array 1.0: 15 auto-antigens were tested on 80 patients/controls; 5 disease associated targets were identified.
Sajda T., Hazelton J., Patel M., Seiffert-Sinha K. Steinman L., Robinson W. H., Haab B. B., and Sinha A. A. 2016. Multiplexed autoantigen microarrays identify HLA as a key driver of anti-desmoglein and -non-desmoglein reactivities in Pemphigus. PNAS 113(7):1859-64. http://www.pnas.org/content/113/7/1859.long
Sinha, A. A. and Sajda, T. 2018. The evolving story of autoantibodies in Pemphigus vulgaris: development of the “super compensation hypothesis”. Front. Med. 5:218. doi: 10.3389/fmned.2018.00218 [Epub ahead of print].

Array 2.0: 50 auto-antigens were tested on 675 patients/controls; 35 disease-associated targets were identified.

TABLE 1

| | Antigens | Samples Screened |
|---|---|---|
| Array 1.0 | 15 | 80 (40 patient, 40 controls) |
| Array 2.0 | 50 | 675 (459 patient, 216 control) |

Reactivities were stratified by clinical subtypes, with static parameters such as age, sex, HLA expression and disease onset, and with dynamic parameters such as disease activity, morphology, and disease duration.

See Sajda, T et al. Proc Natl Acad Sci. 2016 Feb. 16; 113(7):1859-64.

IgG Reactivity was compared for PV patients vs. controls. Thirty five antigens were identified with significantly increased IgG autoreactivity in the PV group. These auto-antigens are shown in Table 2.

TABLE 2
AUTO-ANTIGENS

| ATP2c1 | CHRNE | Dsg1 | HLA E | CHRM3 | ANXA9 | DSC3 |
|---|---|---|---|---|---|---|
| (ATPase | (Cholinergic | (Desmoglein | | (cholinergic | (Annexin A9) | (Desmocollin- |
| Secretory | Receptor | 1) | | receptor | | 3) |
| Pathway Ca2+ | Nicotinic | | | muscarinic 3) | | |
| Transporting 1) | Epsilon | | | | | |
| | Subunit) | | | | | |
| CD33 | Chromatin | Dsg3 | HLA-DRA | CHRM4 | C5a1 | PG |
| | | (Desmoglein | | (cholinergic | | |
| | | 3) | | receptor | | |
| | | | | muscarinic 4) | | |
| CENP A/B | COL3A1 | FcIgE | HSP60 | CHRM5 | COL21A1 | SOD2 |
| | | | | (cholinergic | (collagen type | (superoxide |
| | | | | receptor | XXI alpha 1 | dismutase 2) |
| | | | | muscarinic 5) | chain) | |
| CHRNA9 | Collagens | FH (fumarase) | CHRM1 | NDUFS1 | DSC1 | TG |
| | | | (cholinergic | (NADH- | (Desmocollin- | (thyroglobulin) |
| | | | receptor | ubiquinone | 1) | |
| | | | muscarinic | oxidoreductase) | | |
| | | | 1) | | | |
| CHRND | CYB5B | GP1BA | CHRM2 | PDHA1 | DSC2 | TPO (Thyroid |
| (Acetylcholine | (Cytochrome | (Glycoprotein | (cholinergic | (pyruvate | (Desmocollin- | Peroxidase) |
| receptor | B5 Type B) | Ib Platelet | receptor | dehydrogenase | 2) | |
| subunit delta) | | Subunit | muscarinic | E1 subunit | | |
| | | Alpha) | 2 | alpha 1) | | |

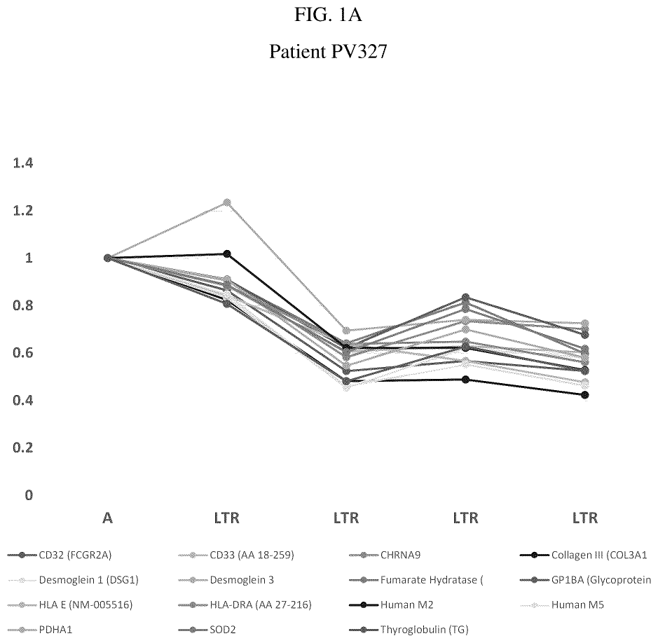

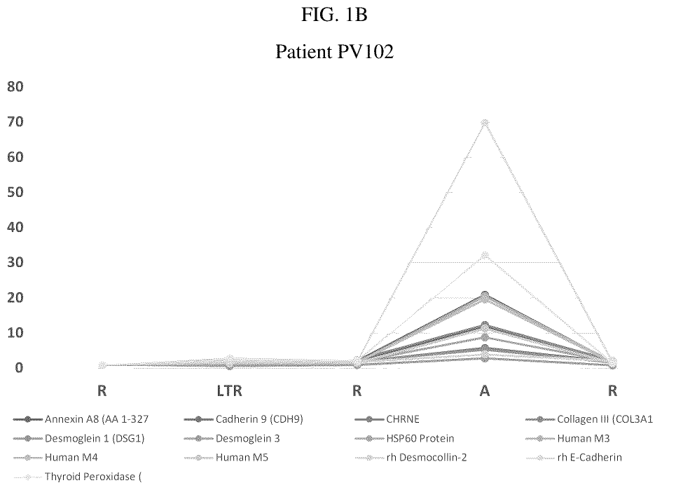

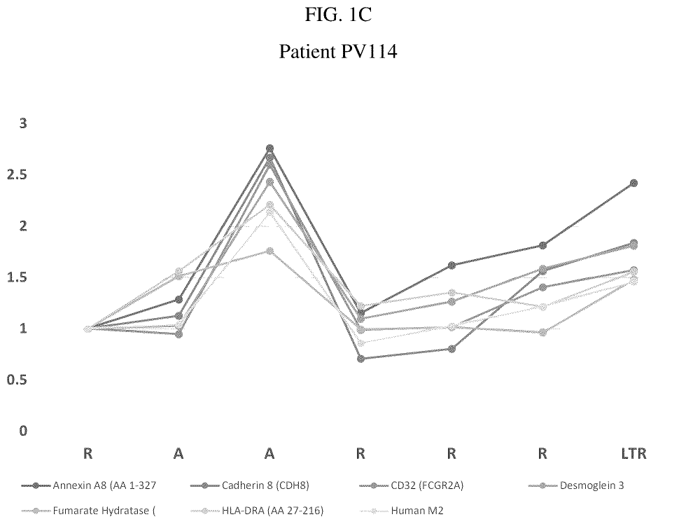

Multiple non Dsg3 and Dsg1 auto-antibodies were found to be correlated with disease activity. The pattern is similar in each patient, with an average of 9 auto-antigens. It appears that the set of antigens driving disease activity differs in each patient. Individual patients have unique auto-antigenic profiles. See FIGS. 1A, 1B, and 1C.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A multi-target therapeutic molecule comprising a T-cell binding moiety linked with a first linker to one or more auto-antigen(s).

2. The multi-target molecule of claim 1, wherein the T-cell binding moiety is an anti-CD3 antibody or an antigen binding fragment thereof.

3. The multi-target molecule of claim 2, wherein T-cell binding moiety is an anti-CD3 antibody or an antigen binding fragment thereof and the anti-CD3 antibody or an antigen binding fragment thereof or the auto-antigen is further linked with a second linker to an anti-CD19 or anti-CD20 antibody, or antigen-binding fragment thereof.

4. The multi-target molecule of claim 1, wherein multiple linkers link the anti-CD3 antibody or an antigen binding fragment thereof to multiple auto-antigens.

5. The multi-target molecule of claim 1, wherein the auto-antigen is Dsg3, Dsg1, or both.

6. The multi-target molecule of claim 1, wherein the auto-antigen is bullous pemphigoid antigen 180, bullous pemphigoid antigen 230, or both.

7. The multi-target molecule of claim 1, wherein the auto-antigen is muscle specific tyrosine kinase (MuSK).

8. The multi-target molecule of claim 1, wherein the auto-antigen is an anti-phospholipase A2 Receptor.

9. The multi-target molecule of claim 1, wherein the antigen binding fragment of an anti-CD3 antibody is a single chain antibody, a Fab fragment, a Fab′ fragment, a F(ab′)2 fragment, or a single chain Fv fragment.

10. The multi-target molecule of claim 1, wherein the anti-CD3 antigen binding fragment of an anti-CD3 antibody is a fragment of an IgG molecule.

11. The multi-target molecule of claim 3, wherein the anti-CD3, anti-CD19, and/or anti-CD20 antibody, or antigen-binding fragment thereof, has reduced or no fucose moieties.

12. A pharmaceutical composition comprising a therapeutically effective amount of a multi-target therapeutic molecule of claim 1.

13-32. (canceled)
